(19) United States
(12) Patent Application Publication
(10) Pub. No.: US 2025/0241631 A1
(43) Pub. Date: Jul. 31, 2025

(54) SYSTEM AND METHOD FOR REAL-TIME SURGICAL NAVIGATION

(71) Applicant: Robotron Technologies Inc., Miami, FL (US)

(72) Inventor: Krzysztof SIEMIONOW, Chicago, IL (US)

(21) Appl. No.: 19/037,469

(22) Filed: Jan. 27, 2025

Related U.S. Application Data
(63) Continuation of application No. 18/897,046, filed on Sep. 26, 2024.
(60) Provisional application No. 63/747,284, filed on Jan. 1, 0001.
(60) Provisional application No. 63/625,931, filed on Jan. 27, 2024.
(60) Provisional application No. 63/687,258, filed on Aug. 26, 2024.

(51) Int. Cl. A61B 17/00; A61B 90/00; A61B 90/30; G06T 7/00; G06T 7/10

(52) U.S. Cl. CPC A61B 17/00234; A61B 90/06; A61B 90/30; A61B 90/361; A61B 90/37; G06T 7/0012; G06T 7/10; A61B 2017/00017; A61B 2017/00075; A61B 2017/00734; G06T 2207/10016; G06T 2207/20084

(57) ABSTRACT

A method for real-time surgical navigation, including: capturing an intraoperative image stream from a distal portion of an endoscopic instrument inserted into a patient's body; processing the intraoperative image stream with a machine learning-based segmentation algorithm configured to identify anatomical structures; matching segmented images to a patient-specific three-dimensional (3D) model of the anatomy; and outputting navigational data indicating the position of the endoscopic instrument relative to the anatomical structures.

### TECHNICAL FIELD

The present invention relates generally to systems and methods for performing minimally invasive surgical procedures with enhanced visualization, particularly in anatomically constrained regions such as the spinal canal. More specifically, this invention addresses real-time image processing techniques, machine learning-based segmentation, and three-dimensional model matching for improved endoscopic navigation and surgical guidance.

### BACKGROUND

Endoscopic techniques have played a transformative role in modern medicine, offering minimally invasive access to a variety of anatomical regions. By threading a small camera-equipped instrument into the body, clinicians can visualize tissues and structures without the extensive dissections required in traditional open procedures. Despite these advantages, the utility of endoscopy in certain domains has been limited by technical constraints and insufficient real-time feedback, particularly in surgeries requiring detailed access to tight, complex spaces.

One such domain is spinal surgery. Traditionally, surgeons rely on magnification tools such as operating microscopes or head-mounted loupes, often coupled with a headlight to illuminate the surgical field. While this equipment can enhance visibility in straightforward cases, it proves less effective for deep or angled regions. In fact, these magnification systems, much like rigid endoscopes, cannot easily provide detailed views “around corners” or through narrow canals within the spinal column, such as the neuroforamina. As a result, critical steps are frequently performed by feel rather than by direct visualization.

This lack of clear vision in challenging areas poses significant risks and uncertainties. Surgeons are sometimes compelled to perform tissue removal “blindly,” increasing the likelihood of accidental damage to nerves or other sensitive structures. Current imaging modalities do not resolve these limitations. Their two-dimensional views offer no direct insight into soft tissue morphology or the detailed anatomy behind bony overhangs. Rigid endoscopic cameras, while an improvement over direct line-of-sight tools, often remain too bulky to navigate effectively within the spinal canal and still cannot visualize tissue hidden by bony contours.

Surgeons often have no quantitative metric to determine precisely how much bone should be removed for adequate decompression, or how to correlate preoperative MRI findings with the real-time intraoperative landscape.

In short, conventional endoscopic technology and imaging approaches have not yet resolved the need for thorough, real-time visualization in spinal surgery. They also fail to provide data-driven guidance that could significantly improve surgical outcomes. The present invention seeks to fill these gaps by introducing a system capable of delivering detailed intraoperative views of hard-to-reach areas, while simultaneously offering an intelligent analysis of relevant anatomical and tissue characteristics.

### SUMMARY

The present invention introduces a comprehensive system and method for enhancing spinal surgery through real-time imaging and AI-driven segmentation. The disclosed system integrates endoscopic imaging, machine learning-based segmentation, and patient-specific 3D model matching to facilitate highly accurate and minimally invasive surgical navigation. By continually aligning intraoperative images from an endoscopic instrument with a segmented 3D model of the patient's anatomy, the system provides real-time positional feedback to surgeons or robotic systems, thereby improving both precision and safety.

By capturing and storing high-quality imaging data from each procedure, the system builds a robust and growing dataset that can refine its algorithms over time, ensuring continuous improvement and accuracy in future cases. In parallel, these data-driven features help lay the groundwork for robotic systems to eventually perform complex surgical interventions with minimal human oversight, as the foundation of precise segmentation, landmark recognition, and three-dimensional anatomy mapping is already in place.

The incorporation of a dual-camera, stereoscopic vision module significantly enhances depth perception and spatial orientation, offering a clear advantage over conventional endoscopic systems that cannot effectively visualize “around corners” or beneath bony structures. Through stereoscopic imaging, surgeons can track instrument movement more accurately, monitor changing volumes in real time, and measure critical anatomical parameters such as endplate distance or disc space volume.

In one aspect, the invention relates to a method for real-time surgical navigation, comprising capturing an intraoperative image stream from a distal portion of an endoscopic instrument inserted into a patient's body, processing the intraoperative image stream with a machine learning-based segmentation algorithm to identify anatomical structures, matching the segmented images to a patient-specific three-dimensional (3D) model of the anatomy, and outputting navigational data indicating the position of the endoscopic instrument relative to the anatomical structures. This allows accurate and dynamic tracking of surgical instruments, thereby enhancing procedural safety and precision.

In a preferred embodiment, the method further comprises applying an encoder-decoder neural network that includes an encoder portion extracting feature maps from each image frame and a decoder portion reconstructing the segmented output identifying specific anatomical structures. Such configuration provides robust and adaptive segmentation, therefore improving recognition reliability under varying intraoperative conditions.

In another preferred embodiment, the method further comprises acquiring stereoscopic image data from two cameras located at the distal portion of the endoscopic instrument and using said stereoscopic image data to estimate a depth map of the operative field. This allows enhanced 3D perception and therefore improves surgical navigation in confined or geometrically complex spaces.

In a further preferred embodiment, the method includes combining the depth map with the patient-specific 3D model to measure at least one of: a distance between the endoscopic instrument and a selected anatomical landmark, an angle or orientation of the endoscopic instrument relative to the anatomy, or a real-time volume of an anatomical region. Therefore, it is possible to make quantitative assessments in real time, thereby facilitating precise surgical planning and execution.

In another preferred embodiment, the method further comprises aggregating multiple consecutive segmented frames to refine the matching of the intraoperative images against the 3D model. This allows stable tracking and alignment over time, thus reducing misregistration caused by intermittent occlusions or lighting variations.

In a further preferred embodiment, the method comprises calibrating the location of the endoscopic instrument relative to the patient's anatomy by referencing one or more known anatomical landmarks or calibration markers within the surgical field. Such configuration provides a reliable reference framework, making it possible to achieve high positional accuracy during navigation.

In another preferred embodiment, the method further comprises controlling, via a robotic manipulator, the position or orientation of the endoscopic instrument in response to the navigational data, thereby enabling semi-autonomous or autonomous navigation within the patient's anatomy. This allows reduced manual workload and consistent motion control, thereby minimizing potential surgical errors.

In another preferred embodiment, the method comprises storing segmented frames and their corresponding location data for post-operative review, wherein said stored data are used to retrain the machine learning-based segmentation algorithm. Such configuration provides an adaptive learning loop, making it possible to continuously enhance segmentation accuracy and system performance over time.

In a further preferred embodiment, the method uses a patient-specific 3D model derived from at least one of a computed tomography (CT) scan or a magnetic resonance imaging (MRI) scan, with bony structures, nerves, intervertebral discs, and other soft tissues segmented. This allows highly individualized navigation guidance, therefore reducing the risk of damaging patient-specific critical structures.

In another preferred embodiment, the method further comprises displaying a color-coded overlay of identified anatomical structures on a monitor or head-mounted display to provide real-time visual feedback to a surgeon or operator. Such configuration provides immediate recognition of critical anatomy, thereby improving procedural awareness and reducing surgical risk.

In a further preferred embodiment, the endoscopic instrument comprises a surgical tool selected from the group consisting of a dissector, nerve hook, rongeur, scissors, or ball probe. This allows both visualization and manipulation through a single device, therefore enhancing efficiency and control during the procedure.

In another preferred embodiment, the machine learning model is trained on a dataset combining real surgical images and synthetic images obtained from physical or virtual 3D-printed anatomical models. This configuration provides a diverse training foundation, making it possible to achieve more robust tissue recognition across varied intraoperative scenarios.

In a further preferred embodiment, the method leverages the patient-specific 3D model data to improve segmentation accuracy of the machine learning-based segmentation algorithm. This allows cross-referencing between preoperative anatomical details and live images, thus yielding higher-fidelity identification of features under real-time conditions.

In another aspect of the invention, a system for real-time surgical navigation is provided, comprising: an endoscopic instrument having a distal portion configured to capture an intraoperative image stream; a computing device that includes one or more processors, memory, and optionally a graphics processing unit; a machine learning-based segmentation module adapted to identify anatomical structures in the intraoperative image stream; and a model matching module that aligns the segmented images with a patient-specific 3D model, producing navigational data. Such configuration provides a comprehensive hardware and software framework, thereby enabling real-time instrument localization and informed surgical guidance.

In a preferred embodiment, the system comprises two cameras at the distal portion of the endoscopic instrument for stereoscopic imaging, enabling the computing device to estimate a depth map of the operative field. Therefore, it is possible to perceive three-dimensional relationships in confined anatomical corridors, thus improving accuracy in surgical maneuvers.

In another preferred embodiment, the system further includes a robotic manipulator communicatively coupled to the computing device, wherein the model matching module provides location and orientation data for semi-autonomous or autonomous positioning of the endoscopic instrument. This allows coordinated and precise movements under automated control, thereby reducing operator fatigue and potential human error.

In a further preferred embodiment, the system incorporates an encoder-decoder neural network as the machine learning-based segmentation module. Such configuration provides efficient and real-time segmentation, therefore maintaining continuous tissue identification even under challenging conditions.

In another preferred embodiment, the system further comprises a user interface module adapted to display a real-time overlay of segmented anatomical structures on a visualization module and to output navigational prompts or warnings. This allows immediate intraoperative feedback, thereby enhancing situational awareness and supporting safer surgical actions.

In a further preferred embodiment, the system employs a patient-specific 3D model derived from CT or MRI scans, stored in a data/model storage and segmented to distinguish relevant anatomical features. Such configuration provides individualized data-driven guidance, making it possible to tailor each surgical procedure to the patient's unique anatomical variations.

In another preferred embodiment, the system comprises a training and update engine configured to store intraoperative image data and segmentation results, and to retrain or fine-tune the machine learning-based segmentation module based on post-operative or offline analysis. This allows continuous improvement of the system's algorithms over multiple procedures, thus advancing surgical efficacy and safety outcomes.

These and other features, aspects and advantages of the invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 illustrates an exemplary endoscopic instrument in accordance with one embodiment.

FIG. 2A provides a hardware-level block diagram of the system architecture in accordance with one embodiment.

FIG. 2B depicts a functional diagram of the software components in accordance with one embodiment.

FIG. 3A shows an example of an intraoperative endoscopic image frame before segmentation, and FIG. 3B shows a corresponding patient-specific 3D model (e.g., a segmented spinal column).

FIG. 4 is a flowchart illustrating how incoming frames from the endoscopic camera are processed by the segmentation algorithm in accordance with one embodiment.

FIG. 5A shows an example of a segmented intraoperative frame with colored boundaries indicating different anatomical structures, FIG. 5B shows an example of 3D model alignment demonstrating matched landmarks, FIG. 5C shows an example depth-based measurement generated through stereoscopic imaging, FIG. 5D shows an example of knee segmentation during arthroscopy and FIG. 5E shows an example of abdominal segmentation.

FIG. 6 is a workflow diagram summarizing a representative endoscopic procedure in accordance with one embodiment.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention.

Minimally invasive surgical procedures, particularly those involving the spinal column or other anatomically intricate regions, often require precise guidance to avoid critical structures and to ensure effective treatment. Traditional surgical navigation methods rely on external reference frames, fluoroscopy, or two-dimensional images, which can limit the surgeon's ability to accurately visualize deep or complex anatomy. Moreover, such methods typically offer static or near-static images that must be mentally correlated by the operator during the procedure.

One or more embodiments of the present invention address these limitations by providing a system and method for real-time imaging and navigation within a patient's anatomy. In particular, the invention leverages an endoscope or integrated surgical instrument equipped with a camera (or cameras) and optional light source to capture live images of the operative field. These intraoperative images are analyzed through a machine learning system, such as an encoder-decoder type deep neural network, trained to identify, segment, and match anatomical structures in the captured images to a patient-specific three-dimensional (3D) model derived from preoperative imaging data (e.g., CT or MRI scans). By continually mapping the real-time images to the 3D model, the system provides surgeons with a dynamic representation of both the instrument's location and the surrounding anatomical structures.

Beyond simply displaying the operative field, the disclosed system is designed to segment and measure critical anatomical features, offering clinicians quantitative data to guide surgical decision-making. In some embodiments, the same segmentation and positional data can be used for autonomous or semi-autonomous navigation, wherein a robotic controller adjusts instrument positioning based on real-time analysis of the patient's anatomy. As a result, surgical efficiency and accuracy are improved while the risk of inadvertent damage to delicate structures, such as nerves in the spinal canal, is reduced.

By employing multi-frame analysis, the system refines its spatial registrations, ensuring robust alignment even under challenging conditions such as partial occlusions, low lighting, or patient-specific anatomical variations. Furthermore, each new surgical procedure can optionally enhance the system's performance over time through continuous learning, wherein annotated intraoperative images are incorporated into the training dataset, further improving the accuracy of future navigation procedures.

While the primary focus herein is directed to spinal surgery, one or more embodiments of the invention can be adapted for use in other anatomical regions, such as the shoulder, elbow, or knee. Moreover, the same principles are applicable to intra-abdominal endoscopic (laparoscopic) procedures, where real-time segmentation can facilitate safer dissection around organs, vessels, or other critical structures. By leveraging similar principles of endoscopic visualization and machine learning-based segmentation, clinicians can navigate these regions with similar benefits of real-time feedback and precise navigation.

The sections that follow describe the components, operation, and use cases of this navigation system in greater detail, emphasizing various hardware, software, and procedural embodiments. It is intended that the invention cover additional embodiments and variations, as would be apparent to those skilled in the art, in view of the disclosures set forth herein.

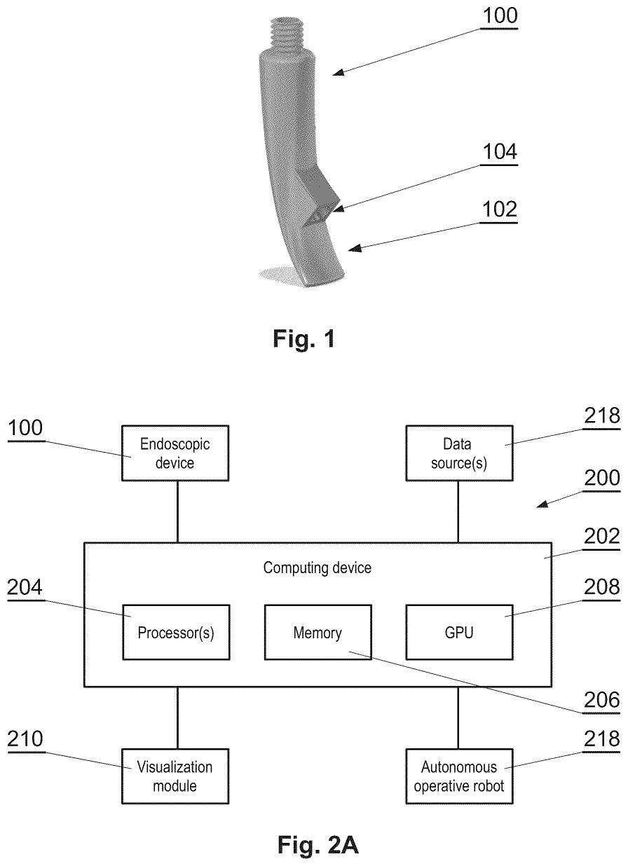

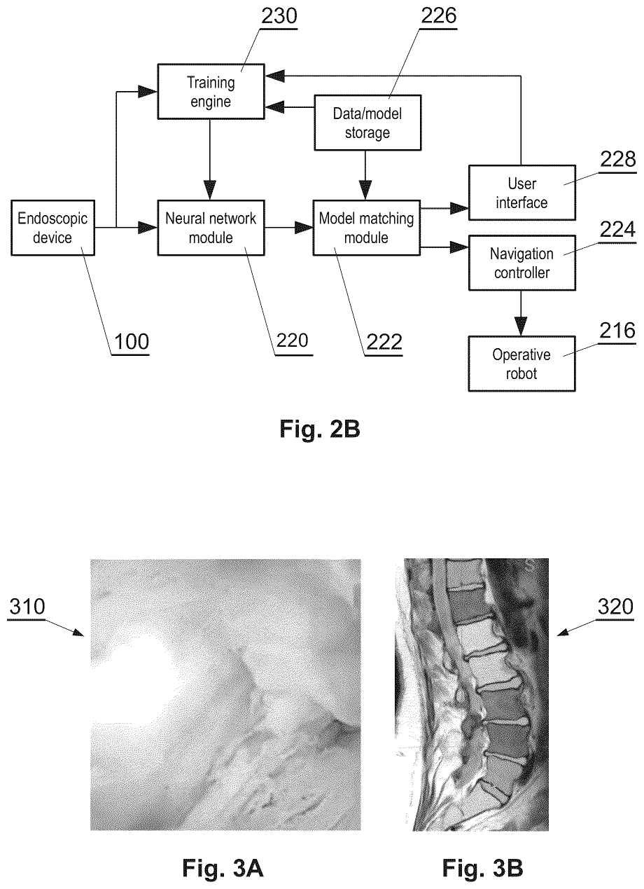

An overview of the system architecture and components will be explained with reference to FIG. 1 that illustrates an exemplary endoscopic instrument 100, and FIG. 2 that provides a high-level block diagram of the overall system architecture 200.

As shown in FIG. 1, the endoscopic instrument 100 includes a distal portion 102 designed for insertion into the patient's body (e.g., through the sacral hiatus or neural foramina) and a proximal portion manipulated by the surgeon or a robotic apparatus. The distal portion 102 comprises a vision module 104 combined with the body of the instrument, which may include one or more cameras having a field of view that covers the vicinity of the distal portion 102, as well as lighting directed toward that field of view. In certain embodiments, the vision module 104 houses two cameras 106L, 106R, arranged to capture stereoscopic image data for real-time depth estimation. This arrangement enables more accurate distance and location measurements of the observed anatomy.

The camera(s) may be a high-definition (HD) optical sensor or another suitable imaging device. In embodiments employing two cameras 106L, 106R, the system can generate a stereoscopic signal to derive depth information in the field of view. The light source can be one or more light-emitting diodes (LEDs), positioned adjacent to each camera to provide adequate illumination of the surgical field. In some embodiments, multispectral or infrared lighting is optionally included to enhance tissue differentiation if desired. When the distal portion 102 includes two cameras 106L, 106R, their outputs are combined by the computing device 202 to create a stereoscopic depth map, allowing the system to calculate the distance of each visible anatomical structure from the instrument 100. This stereoscopic approach can further refine the model matching process by providing additional spatial cues, improving the accuracy of instrument localization and tissue segmentation even in narrow or partially occluded regions of the operative field.

In further embodiments, the endoscopic instrument 100 takes the form of a surgical tool such as a 90° dissector, 90° ball probe, nerve hook, curved scissors, pituitary rongeur, Woodson dissector, Penfield dissector, or any other suitable device that combines a functional tip with the vision module 104. This configuration allows tissue manipulation and imaging to occur simultaneously, facilitating close monitoring of anatomical structures as they are dissected or retracted. Alternatively, the endoscope can be a standalone device introduced alongside one or more separate surgical instruments.

To facilitate passage into the spinal canal (or another target anatomy), the distal portion 102 of the endoscopic instrument 100 is typically slender and may include steering or angulation capabilities. Depending on the embodiment, the lens of the camera can be optimized for wide-angle viewing in the constrained surgical space. Although FIG. 1 depicts an exemplary configuration, numerous variations are possible—for instance, a smaller-diameter instrument may be preferred in pediatric or delicate contexts.

Turning to FIG. 2A, a hardware-level block diagram of the system architecture 200 is shown. A computing device 202 has one or more processors 204, memory 206, and optionally a graphics processing unit (GPU) 208 or other hardware accelerators for real-time image processing. The computing device 202 communicates with the other hardware components via wired or wireless connections. A visualization Module 210, such as a display screen 212, an augmented reality (AR) headset, or a virtual reality (VR) device, displays the real-time operative view, overlay annotations, and patient-specific 3D model information to the surgeon.

Optionally, the computing device 202 may be coupled with an autonomous operative robot 216 with a robotic manipulator or arm that can hold, position, or manipulate the endoscopic instrument 100 or other surgical tools and that receives control signals from the computing device 202 based on real-time navigation data.

The endoscopic device 100, as previously described with reference to FIG. 1, captures intraoperative images and transmits them to the computing device 202.

The system also comprises external data source(s) 218 that store or provide preoperative 3D models derived from patient scans (e.g., CT, MRI). The data may be accessed locally or over a network, enabling the computing device 202 to retrieve and load the appropriate model for a given procedure.

Communication links in FIG. 2A indicate the flow of data among these hardware components. For instance, the endoscopic device 100 transmits live video to the computing device 202, which in turn processes the images and sends graphical overlays to the visualization module 210. If robotic assistance is enabled, the computing device 202 also sends control signals to the autonomous operative robot 216.

FIG. 2B illustrates the functional or software-level components within the system architecture 200. These modules typically execute on or in conjunction with the computing device 202, although some functionalities could be distributed across multiple devices or servers in a cloud environment.

A neural network module 220 implements a machine learning or AI-based system, such as a convolutional neural network (CNN). It receives image frames from the endoscopic device 100 and performs segmentation, object recognition, or other image analysis tasks. An example of an image frame 310 before segmentation is shown in FIG. 3A.

A model matching module 222 aligns the segmented images from the neural network module 220 with a patient-specific 3D model. It determines the relative position and orientation of the endoscopic instrument 100 by matching detected anatomical landmarks in the live images to their corresponding structures in the 3D model. An example visualization of a 3D spine model 320 (arranged on top of a CT scan) is shown in FIG. 3B, with various sections marked therein by different shades.

A navigation controller 224 uses positional and orientation data from the model matching module 222 to guide the user or an autonomous robot. For example, in autonomous embodiments, it may send operative instructions or motion commands to the autonomous operative robot 216. In manual modes, it may generate visual and/or audible prompts to be presented by the visualization module 210 to assist the surgeon in navigating the surgical field.

A data/model storage 226 is a repository (e.g., a database) for maintaining the 3D anatomical models, segmentation maps, and any intermediate or long-term data needed for training or refining the neural network module 220. It may also store patient records, procedure logs, or calibration data.

A user interface module 228 communicates with the visualization module 210 to render real-time overlays, color-coded segmentations, text-based alerts, or other user-friendly guidance. Integrates features such as measurement displays, highlighting of critical anatomy structures, and interactive controls for the operator.

A training and update engine 230 aggregates intraoperative data and user feedback for continuous learning or post-operative AI model refinement. It may operate offline or in the background to update the neural network module 220 with new cases or improved annotations.

In operation, the Neural Network Module 220 processes each incoming frame from the vision module 104, returning segmentation results to the model matching module 222, which aligns them with the 3D model. The navigation controller 224 then uses this alignment data to display navigational cues to the surgeon (via the user interface module 228) or to control the autonomous operative robot 216. This real-time loop enables enhanced surgical navigation, whether fully manual, semi-autonomous, or robotic-controlled.

In view of the above components, the following section will describe how the 3D model is obtained and segmented for use by the system's functional components. Preoperative generation of a patient-specific, three-dimensional (3D) model is key to the surgical navigation system described herein. By converting patient imaging data—such as a computed tomography (CT) or magnetic resonance (MRI) scan—into a segmented 3D representation of the target anatomy, the system can accurately align intraoperative endoscopic images with the patient's specific anatomical structures.

In one embodiment, the patient undergoes a CT or MRI scan focused on the region of interest (e.g., the lumbar spine). These volumetric images are then stored in standard medical imaging formats (e.g., DICOM) at the external data source 218. Once acquired, the volumetric datasets are processed by commercial or custom software to produce the 3D model. This model can be rendered as a mesh (e.g., STL format) or as a volumetric grid that preserves interior structural data. After the basic 3D reconstruction is obtained, the applies one or more segmentation techniques can be applied to label relevant tissues. For instance, bony structures may be separated from neural elements and soft tissues. The resulting segmented model may be provided from the external data source 218 and stored in the data/model storage 226 with each structure assigned a label. This labeled 3D model can include bony regions, neural regions (e.g., nerve roots), discs, and other soft tissue structures critical for surgical navigation.

Once the segmented 3D model is stored in the data/model storage 226, it becomes accessible to the neural network module 220 and the model matching module 222 during the following procedures. Prior to surgery, the computing device 202 retrieves the patient-specific 3D model from the external data source 218 or from data/model storage 226 if pre-loaded. The model is initialized within the model matching module 222, which will later align intraoperative images to the corresponding anatomical references.

In some embodiments, the system may use known landmarks or calibration methods to ensure the 3D model's coordinate system aligns with that of the actual patient. The surgeon or a robotic manipulator (if present) can then rely on this alignment for precise instrument navigation.

While the neural network module 220 focuses on intraoperative image segmentation, the preoperative segmentation of the 3D model offers a complementary dataset for matching shapes and features. This synergy between preoperative and intraoperative data enhances the accuracy of tissue identification—particularly in cases of partial occlusion or suboptimal lighting during surgery.

The use of a segmented 3D model ensures that the system has a comprehensive map of the patient's anatomy before entering the operating room. This preoperative map provides several benefits. It allows for accurate tissue differentiation—nerves, discs, and bony landmarks are explicitly labeled, reducing the risk of accidental instrument contact with critical structures. Furthermore, it allows quantitative analysis, such that dimensions, e.g. disc height or canal diameter, can be measured on the preoperative model and compared to intraoperative findings in near real-time. Furthermore, by matching the real-time images from the endoscopic instrument 100 to the patient's model, the system continuously updates the display to show the surgeon's or robot's position relative to labeled structures.

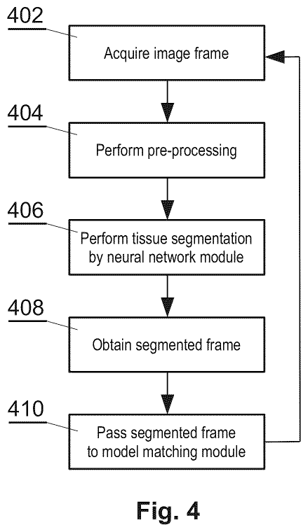

The system employs a machine learning approach to process real-time images captured by the endoscopic instrument 100, as detailed in the flowchart of FIG. 4. In particular, the neural network module 220 is responsible for segmenting each incoming frame and generating labeled outputs used by the model matching module 222 to align the live view with a preoperative 3D model. The primary steps in this process are described below. Referring to FIG. 4, at step 402, the system acquires a new frame from the endoscopic instrument 100. Preprocessing of the frame (e.g., noise reduction, color normalization) occurs at step 404, preparing the data for efficient handling by the encoder-decoder neural network. At step 406, the neural network module 220 applies a trained encoder-decoder architecture to perform tissue segmentation and extract relevant anatomical features. The resulting labeled (segmented) frame is obtained at step 408, preferably accompanied by confidence scores or bounding contours for each identified structure. In step 410, the labeled frame is then passed to the model matching module 222 to be compared against the patient's preoperative 3D model. Finally, the workflow returns to step 402 to receive the next frame in a continuous loop.

The model used at step 406 typically follows an encoder-decoder neural network architecture (e.g., a U-Net variant), which allows efficient feature extraction across different spatial scales and subsequent upsampling to produce segmentation masks. While convolutional layers are commonly utilized within such encoder-decoder frameworks, the invention is not limited to strictly CNN-based models and may employ other deep learning paradigms. The GPU 208 in the computing device 202 can substantially reduce inference latency, ensuring that step 406 keeps pace with the real-time video stream.

The encoder-decoder network should be trained to handle environmental variability (lighting shifts, occlusions) likely to appear at step 402. Optional pre-processing at step 404 may include smoothing or color balancing to compensate for shadows or fluid in the field of view.

The neural network module 220 is trained on a diverse dataset comprising both real surgical footage and 3D-printed model imagery. For example, real surgical videos can be used to provide authentic anatomical variance and intraoperative complexity, while 3D-printed model footages offer consistent, controlled scenarios to reinforce basic shape recognition.

During training, each frame is annotated with ground-truth labels. The system subsequently refines or updates these learned parameters post-operatively, or using data collected from other procedures. Such updates might be orchestrated by the optional training and update engine 230.

The neural network module 220 may be configured to process single frames or multiple frames. In the single-frame scenario, each image may enter the encoder-decoder pipeline at steps 402-406 independently, enabling immediate classification of visible structures. In a multi-frame context, the system may aggregate consecutive labeled frames, reducing noise and improving reliability in challenging scenes (e.g., partial occlusions). Optional smoothing or filtering can be applied between steps 408 and 410, stabilizing segmentation masks over time and preventing flicker in the displayed overlay.

Thus, the procedure allows for real-time adaptability, as steps 402-406 are repeated at video frame rate, enabling continuous updates for navigation. By highlighting sensitive anatomical structures, the surgeon or robotic controller (if present) obtains precise information on current location and can avoid sensitive tissues.

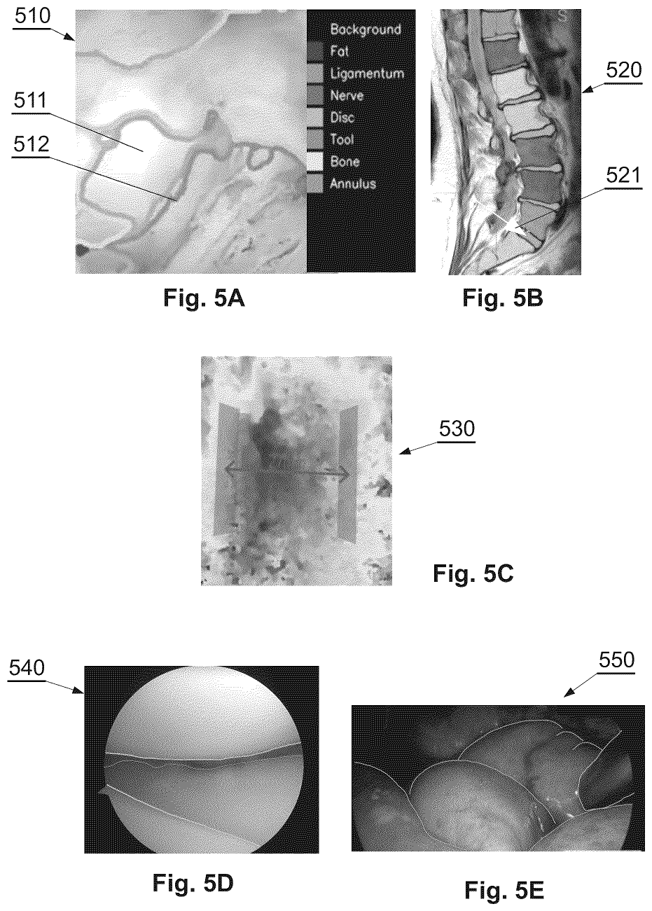

The model matching process is performed repeatedly, frame by frame, to ascertain the precise location and orientation of the endoscopic instrument 100 (or any attached surgical tool) within the patient's anatomy. An exemplary depiction of this matching process is shown in FIG. 5. In general, the model matching module 222 receives segmented frames (images from the neural network module 220, wherein each pixel (or region) is labeled e.g. as bone, disc, nerve, or other tissue class) and preoperative 3D model, which is a patient-specific, segmented 3D anatomy stored in the data/model storage 226 and loaded into the computing device 202 at the start of the procedure. Based on these inputs, the model matching module attempts to establish correspondences between features (e.g., bony contours, ligament outlines, nerve paths) in the segmented frame and those in the 3D model. By computing the transformation that best aligns these recognized structures, the system determines the position and orientation of the vision module 104 relative to the patient's anatomy. To do that, the model matching module 222 searches for distinctive landmarks—such as edges of vertebral bodies, disc boundaries, or the branching points of nerve roots—that are identifiable in both the segmented intraoperative frame and the 3D model. Once landmarks are identified, the module can apply geometric matching or registration algorithms to reduce any discrepancy between the image-derived landmarks and the 3D model geometry. A matching score or error metric can be calculated, and the alignment can be refined iteratively until the best fit is found. Because certain structures may be hidden by tissue, surgical tools, or blood, the module may only match a subset of landmarks at any given frame. However, the module can still reliably estimate the instrument's location by combining recognized landmarks over several consecutive frames and filtering outliers.

As new frames arrive from the neural network module 220 in step 406 of FIG. 4, the model matching module 222 can re-evaluate or refines the previously determined alignment, by incremental tracking—having established an initial position, the module can predict the instrument's likely movement in the 3D model, thus speeding up subsequent registration. By aggregating data across multiple frames, the system can maintain stable tracking even if one frame is low-quality (e.g., blurred or partially occluded).

Once the model matching module 222 determines the instrument's location, that information is passed to the navigation controller 224. If the system is operating in manual mode, the navigation controller 224 may provides guidance cues (e.g., “move slightly to the left to reach the target disc space”) to the user interface module 228. In autonomous or semi-autonomous modes, the navigation controller 224 may send control signals to the autonomous operative robot 216 to reposition the instrument or perform a predefined task, such as tissue resection.

Furthermore, by means of the user interface module 228, the current location of the endoscopic instrument 100 can be overlaid on the patient-specific 3D model in real time. For example, the operator may see color-coded boundaries on the live video feed to indicate nerve tissues or vital structures, helping to avoid accidental damage. Additional data, such as the distance to a specific anatomical landmark, can be displayed to assist surgical decision-making

FIGS. 5A, 5B, 5C, 5D and 5E show an example user interface, which shows a segmented intraoperative frame 510 (FIG. 5A), with boundaries or color-coded structures 511 overlaid on the real image 512 (e.g., bone in white, disc in red, nerves in yellow) and a 3D model alignment 520 (FIG. 5B), showing how corresponding structures are matched, with a highlighted marker representing the computed location and orientation of the endoscopic instrument. Arrows 521 or lines might illustrate how distinctive landmarks in the intraoperative frame map to their counterparts in the 3D model, demonstrating the registration process in real time. Moreover, FIG. 5C shows an example result 530 of operation of a stereoscopic camera, capable of determining distances between two anatomical structures. FIG. 5D shows an example result 540 of knee segmentation during arthroscopy and FIG. 5E shows an example of result 550 of abdominal segmentation.

The model matching module 222 may compute a confidence score (or registration error) for the alignment. If the score drops below a certain threshold, the system can prompt the operator to perform some adjustments, such as change the endoscopic device position.

The optional training and update engine 230 can record instances where misalignment occurred, using that data post-operatively to refine the matching algorithms or the neural network's segmentation accuracy.

By constantly tracking the instrument within the patient's 3D anatomy, surgeons or robotic systems can perform targeted interventions without guesswork related to the current position within the patient's anatomy. Furthermore, delicate neural elements remain visible and protected, reducing the risk of inadvertent damage.

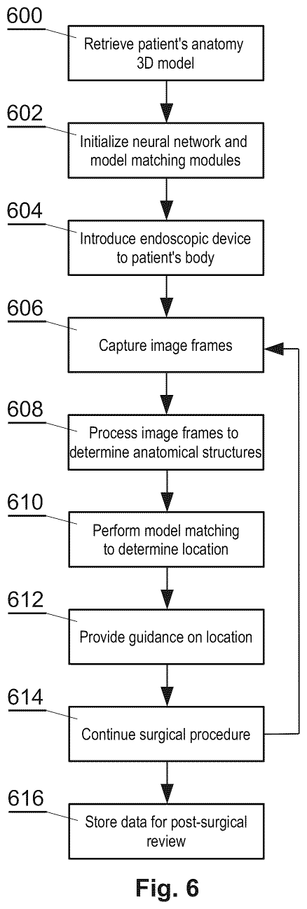

The system described above can be used in an exemplary surgical workflow to guide an endoscopic procedure in real time. Referring to FIG. 6, this workflow can be conceptually divided into initialization, image capture and processing, navigation feedback, and completion. While the example below focuses on spinal surgery, similar principles apply to other anatomical targets (e.g., shoulder, elbow, knee). As shown in FIG. 6, the surgical procedure typically proceeds through the following steps. In step 600, the computing device 202 retrieves the patient's preoperative 3D anatomy model from the external data source 218 or from local data/model storage 226. In step 602, various modules, including the neural network module 220 and the model matching module 222, are initialized and configured. The surgeon or an assistant may also perform a calibration routine to align the real-world coordinate system with the 3D model. In step 604, the endoscopic instrument 100 is introduced into the patient's body (e.g., via the sacral hiatus), ensuring that the vision module 104 has a clear operative view. In step 606, live image frames from the camera are sent to the computing device 202, triggering the real-time analysis pipeline. Next, in step 608 the neural network module 220 processes each frame to identify and label anatomical structures, leveraging pretrained models as discussed with reference to FIG. 4. In step 610 the segmented frames are passed to the model matching module 222, which aligns the identified structures with the preoperative 3D model to determine the instrument's location. The navigation controller 224 uses the matched location data to provide, in step 612, guidance to the user (via the user interface module 228) and/or send control commands to an autonomous operative robot 216 if operating in robotic mode. Based on the real-time navigation feedback, the surgeon or robot manipulates instruments to perform the specific procedures in step 614, continuously receiving updated positional and anatomical cues. Once the procedure is finished, the system stores, in step 616, relevant data (e.g., video footage, instrument trajectories) for post-operative review or future training updates.

During steps 608 and 610, the system makes use of the capabilities of the neural network module 220 and the model matching module 222. If robotic automation is enabled, the navigation controller 224 at step 612 issues commands to the autonomous operative robot 216, ensuring the instrument's movement remains consistent with the real-time anatomical mapping. Alternatively, in manual mode, the surgeon visually tracks navigation cues on a display screen 212 or AR/VR headset 214.

This procedure allows surgeons to benefit from a continuous overlay of live images and 3D model data, enhancing decision-making. Robotic or semi-autonomous procedures can leverage real-time anatomical matching to reduce errors and improve reproducibility. Moreover, by logging each procedure's data, future training can refine segmentation accuracy (neural network module 220) and alignment algorithms (model matching module 222).

While the foregoing sections describe a system particularly suitable for spinal surgery, numerous alternative embodiments and extensions of this invention may be realized without departing from the inventive concepts disclosed herein. For example, arthroscopic procedures could benefit from the same real-time image segmentation and model matching, aiding in rotator cuff repair or labral tear surgery. Similarly, endoscopic or arthroscopic instruments fitted with a vision module 104 can help surgeons navigate narrow joint spaces and preserve neurovascular structures. Furthermore, in procedures such as meniscal repair or anterior cruciate ligament (ACL) reconstruction, intraoperative endoscopic images can be aligned with a 3D model of the femur, tibia, and menisci. Moreover, with some adaptations (e.g., a narrower or angled endoscope), the system may be employed for endonasal approaches in neurosurgery or otolaryngology, aligning real-time views of sinus or skull base anatomy to a corresponding 3D model.

In some embodiments, two or more surgical instruments can be simultaneously tracked. If additional endoscopes or cameras are introduced, each feed can be processed via separate instances of the neural network module 220 or via a shared framework, providing complementary viewpoints of the surgical field. The model matching module 222 can then merge positional data, potentially offering stereoscopic or multi-angle depth perception. In certain configurations, rather than introducing multiple endoscopes, a single endoscopic instrument 100 may incorporate two cameras 106L, 106R for stereoscopic visualization, enabling real-time depth perception and enhanced spatial accuracy.

A robotic manipulator 216 may control one instrument autonomously, while a surgeon manually operates another. The navigation controller 224 can ensure that movement paths are coordinated to avoid collisions and maintain clear visualization.

Although the disclosed embodiments focus on optical endoscopic imaging, the invention can be adapted to integrate or fuse data from additional imaging modalities. For example, a miniaturized ultrasound probe may be combined with or mounted alongside the vision module 104, providing soft tissue detail not always discernible in conventional white-light endoscopy. Furthermore, intraoperative fluorescence markers can help distinguish vascular or malignant tissue, feeding the neural network module 220 for more precise segmentation in certain oncologic or vascular procedures.

In advanced deployments, the system may integrate with electronic health records (EHR) and other clinical data systems. Preoperative planning data (e.g., desired fusion level, specific resection margins) can be annotated in the 3D model, which the model matching module 222 references during the procedure. Data logged from each procedure (e.g., instrument paths, duration, outcomes) can be used to generate metrics for quality improvement and to refine future training datasets.

Although the preceding sections have presented the system in a general manner, the following examples highlight specific procedures and workflows, illustrating how the invention can be employed in real or simulated clinical settings. These examples are illustrative in nature and not intended to limit the scope of the claims.

### Example 1: Lumbar Decompression Surgery

The patient undergoes a lumbar spine MRI, which is converted into a segmented 3D model as described in Section 3. Bony structures, neural elements, and intervertebral discs are labeled for subsequent matching. In the operating room, the computing device 202 loads the patient's 3D lumbar model from the external data source 218. The neural network module 220 is initialized with the relevant trained neural network model parameters for spinal tissue recognition. The surgeon introduces a Penfield-type endoscopic instrument 100 (with vision module 104) through the sacral hiatus. Live images are captured and forwarded to the computing device 202, where each frame is preprocessed and then segmented by the neural network module 220. The model matching module 222 aligns segmented structures (e.g., lamina, ligamentum flavum) with the corresponding regions of the preoperative 3D model, establishing the instrument's location. The navigation controller 224 overlays real-time markers onto the user's display (visualization module 210), guiding the surgeon's approach to the targeted decompression site (e.g., L4-L5 level). The surgeon, aided by clearly visualized nerve roots and bony landmarks, removes compressive tissue while avoiding critical neural structures. Any changes in orientation are instantly reflected in the displayed overlay, allowing continuous instrument tracking and reducing the need for fluoroscopic guidance. At the conclusion, procedure data—instrument trajectories and frame-by-frame segmentation logs—are stored in data/model storage 226 for post-operative review and possible future re-training.

### Example 2: Arthroscopic Shoulder Repair

In this scenario, the same system architecture is adapted for shoulder arthroscopy. A smaller-diameter endoscope 100 with an angled distal portion 102 is employed to visualize the glenohumeral joint. The preoperative MRI of the shoulder is segmented to differentiate humeral head, glenoid, labrum, and rotator cuff structures. During the surgery, frames captured by the vision module 104 depict the arthroscopic view of the joint. The neural network module 220 recognizes cartilage, tendon, and capsular tissues. Despite fluid irrigation and potential bleeding, the segmentation remains robust due to diverse training data that included arthroscopic scenarios and 3D-printed shoulder models. The model matching module 222 aligns the segmented features (e.g., edges of the glenoid or humeral head) to the shoulder's 3D model. As the surgeon manipulates the instrument, the navigation controller 224 continuously updates the overlay on the display, showing the instrument's position relative to critical anatomical landmarks (e.g., the supraspinatus tendon). A robotic suture passer (optionally controlled by the system) may assist in placing sutures precisely where rotator cuff tears are identified. The integrated overlay minimizes misplacement of anchors or sutures, thus enhancing repair accuracy.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method for real-time surgical navigation, comprising:
capturing an intraoperative image stream from a distal portion of an endoscopic instrument inserted into a patient's body;
processing the intraoperative image stream with a machine learning-based segmentation algorithm configured to identify anatomical structures;
matching segmented images to a patient-specific three-dimensional (3D) model of the anatomy; and
outputting navigational data indicating the position of the endoscopic instrument relative to the anatomical structures.

2. The method of claim 1, wherein the machine learning-based segmentation algorithm is an encoder-decoder neural network that comprises an encoder portion for extracting feature maps from each image frame and a decoder portion for reconstructing a segmented output identifying specific anatomical structures.

3. The method of claim 1, further comprising acquiring stereoscopic image data from two cameras located at the distal portion of the endoscopic instrument, and using the stereoscopic image data to estimate a depth map of the operative field.

4. The method of claim 3, further comprising combining the depth map with the patient-specific three-dimensional (3D) model to measure at least one of:
a distance between the endoscopic instrument and a selected anatomical landmark,
an angle or orientation of the endoscopic instrument relative to the anatomy, or
a real-time volume of an anatomical structure.

5. The method of claim 1, further comprising aggregating multiple consecutive segmented frames to refine the matching of the intraoperative images against the patient-specific three-dimensional (3D) model.

6. The method of claim 1, further comprising calibrating the location of the endoscopic instrument relative to the patient's anatomy by referencing one or more known anatomical landmarks or calibration markers within a surgical field.

7. The method of claim 1, further comprising controlling, via a robotic manipulator, the position or orientation of the endoscopic instrument in response to the navigational data, thereby enabling semi-autonomous or autonomous navigation within the patient's anatomy.

8. The method of claim 1, further comprising storing segmented frames and corresponding location data for post-operative review, wherein the stored data are used to retrain the machine learning-based segmentation algorithm and improve its accuracy over subsequent procedures.

9. The method of claim 1, wherein the patient-specific three-dimensional (3D) model is derived from at least one of a computed tomography scan or a magnetic resonance imaging scan, and is segmented to distinguish bony structures, nerves, intervertebral discs, and other soft tissues relevant to a surgical target.

10. The method of claim 1, further comprising displaying a color-coded overlay of identified anatomical structures on a monitor or head-mounted display to provide real-time visual feedback to a surgeon or operator during navigation.

11. The method of claim 1, wherein the endoscopic instrument comprises a surgical tool selected from the group consisting of a dissector, nerve hook, rongeur, scissors, or ball probe.

12. The method of claim 1, wherein the machine learning-based segmentation algorithm is trained on a dataset combining real surgical images and synthetic images obtained from physical or virtual three-dimensional printed anatomical models.

13. The method of claim 1, further comprising using data from the patient-specific three-dimensional (3D) model to improve segmentation accuracy of the machine learning-based segmentation algorithm.

14. A system for real-time surgical navigation, comprising:
an endoscopic instrument having a distal portion configured to capture an intraoperative image stream via an imaging module;
a computing device including one or more processors, memory, and optionally a graphics processing unit;
a machine learning-based segmentation module stored in the memory, the machine learning-based segmentation module being adapted to identify anatomical structures in the intraoperative image stream;
a model matching module configured to align segmented images from the machine learning-based segmentation module with a patient-specific three-dimensional (3D) model of the anatomy; and
an output interface that provides navigational data indicating the position of the endoscopic instrument relative to identified anatomical structures.

15. The system of claim 14, wherein the endoscopic instrument comprises two cameras at the distal portion for stereoscopic imaging, enabling the computing device to estimate a depth map of the operative field.

16. The system of claim 14, further comprising a robotic manipulator communicatively coupled to the computing device, wherein the model matching module provides location and orientation data to the robotic manipulator to enable semi-autonomous or autonomous positioning of the endoscopic instrument.

17. The system of claim 14, wherein the machine learning-based segmentation module is an encoder-decoder neural network.

18. The system of claim 14, further comprising a user interface module adapted to display a real-time overlay of segmented anatomical structures on a visualization module and to output navigational prompts or warnings based on data from the model matching module.

19. The system of claim 14, wherein the patient-specific three-dimensional (3D) model is obtained from at least one of a computed tomography scan or a magnetic resonance imaging scan, is stored in a data or model storage, and is segmented to distinguish anatomical features.

20. The system of claim 14, further comprising a training and update engine configured to store intraoperative image data and segmentation results, and to retrain or fine-tune the machine learning-based segmentation module based on post-operative or offline analysis.
